(19) United States
(12) Patent Application Publication
(10) Pub. No.: US 2005/0214303 A1
(43) Pub. Date: Sep. 29, 2005

(54) Methods for damaging cells using effector functions of anti FAM3D antibodies

(75) Inventors: Yusuke Nakamura, Yokohama-shi (JP); Yataro Daigo, Yokohama-shi (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: Oncotherapy Science, Inc., Minato-ku (JP); Japan as Represented by the President of the University of Tokyo, Bunkyo-ku (JP)

(21) Appl. No.: 10/809,821

(22) Filed: Mar. 24, 2004

(51) Int. Cl. A61K 39/395

(52) U.S. Cl. 424/155.1

(57) ABSTRACT

The present invention relates to the use of cytoxicity based on the effector function of anti-FAM3D antibodies. Specifically, the present invention provides methods and pharmaceutical compositions that comprise an anti-FAM3D antibody as an active ingredient for damaging FAM3D-expressing cells using antibody effector function. Since FAM3D is strongly expressed in lung cancer cells, the present invention is useful in lung cancer therapies.

### TECHNICAL FIELD

The present invention relates to methods for damaging cells using the effector function of anti-FAM3D antibodies, or to compositions for this purpose.

### BACKGROUND ART

Lung cancer is one of the most common lethal human tumors. Non-small-cell lung cancer (NSCLC) is the most common form, accounting for nearly 80% of lung tumors (American Cancer Society, Cancer Facts and Figures 2001, Am. Chem. Soc. Atlanta, 2001). The majority of NSCLCs are not diagnosed until an advanced stage, and thus the overall 10-year survival rate has stayed low at 10%, despite recent advances in multimodality therapies (Fry et al., Cancer, 86: 1867-76, 1999). Currently, chemotherapy using platinum is considered to be a fundamental therapy for NSCLCs. However, the therapeutic action of pharmaceutical agents has not progressed beyond the point of being able to prolong the survival of advanced NSCLC patients to a certain extent (Chemotherapy in non-small cell lung cancer: a meta-analysis using updated data on individual patients from 52 randomized clinical trials, Non-small Cell Lung Cancer Collaborative Group, Bmj. 311: 899-909, 1995). A number of targeting therapies are being investigated, including those that use tyrosine kinase inhibitors. However, to date, promising results have been achieved only in a limited number of patients, and in some patients, therapeutic effects have accompanied severe side effects (Kris et al., Proc Am Soc Clin Oncol, 21: 292a (A1166), 2002).

Research aiming at the elucidation of carcinogenic mechanisms has revealed a number of candidate target molecules for anti-tumor agents. For example, the farnesyltransferase inhibitor (FTI) is effective in the therapy of Ras-dependent tumors in animal models (He et al., Cell, 99: 335-45, 1999). This pharmaceutical agent was developed to inhibit growth signal pathways related to Ras, which is dependant on post-transcriptional farnesylation. Human clinical trials where anti-tumor agents were applied in combination with the anti-HER2 monoclonal antibody trastuzumab with the aim of antagonizing the proto-oncogene HER2/neu have succeeded in improving clinical response, and improved the overall survival rate of breast cancer patients (Lin et al., Cancer Res, 61: 6345-9, 2001). Tyrosine kinase inhibitor STI-571 is an inhibitor which selectively deactivates bcr-abl fusion protein. This pharmaceutical agent was developed for the therapy of chronic myeloid leukemia, where the constant activation of bcr-abl tyrosine kinase has a significant role in the transformation of white blood cells. Such pharmaceutical agents are designed to inhibit the carcinogenic activity of specific gene products (Fujita et al., Cancer Res, 61: 7722-6, 2001). Thus, in cancer cells, gene products with promoted expression are usually potential targets for the development of novel anti-tumor agents.

Another strategy for cancer therapy is the use of antibodies which bind to cancer cells. The following are representative mechanisms of antibody-mediated cancer therapy:

Missile therapy: in this approach a pharmaceutical agent is bound to an antibody that binds specifically to cancer cells, and the agent then acts specifically on the cancer cells. Even agents with strong side effects can be made to act intensively on the cancer cells. In addition to pharmaceutical agents, there are also reports of approaches where precursors of pharmaceutical agents, enzymes which metabolize the precursors to an active form, and so on are bound to the antibodies.

The use of antibodies which target functional molecules: this approach inhibits the binding between growth factors and cancer cells using, for example, antibodies that bind growth factor receptors or growth factors. Some cancer cells proliferate depending on growth factors. For example, cancers dependent on epithelial growth factor (EGF) or vascular endothelial growth factor (VEGF) are known. For such cancers, inhibiting the binding between a growth factor and cancer cells can be expected to have a therapeutic effect.

Antibody cytotoxicity: antibodies that bind to some kinds of antigens can comprise cytotoxicity to cancer cells. With these types of antibodies, the antibody molecule itself comprises a direct anti-tumor effect. Antibodies that display cytotoxicity to cancer cells are gaining attention as antibody agents expected to be highly effective against tumors.

### DISCLOSURE OF THE INVENTION

The present inventors investigated antibodies able to induce cytotoxity, targeting genes showing increased expression in cells. The results revealed that potent cytotoxicity can be induced in FAM3D-expressing cells when those cells are contacted with anti-FAM3D antibodies, thus completing the present invention.

Specifically, the present invention relates to the following pharmaceutical compounds or methods:
[1] a pharmaceutical composition comprising an anti-FAM3D antibody as an active ingredient, wherein the compound is for damaging an FAM3D-expressing cell using the antibody effector function;
[2] the pharmaceutical composition of [1], wherein the FAM3D-expressing cell is a lung cancer cell;
[3] the pharmaceutical composition of [1], wherein the anti-FAM3D antibody is a monoclonal antibody;
[4] the pharmaceutical composition of [1], wherein the antibody effector function is either antibody-dependent cytotoxicity or complement-dependent cytotoxicity, or both;
[5] a method for damaging an FAM3D-expressing cell, comprising the steps of:
a) contacting the FAM3D-expressing cell with an anti-FAM3D antibody, and
b) damaging the FAM3D-expressing cell with the effector function of the antibody that has bound to the cell;
[6] an immunogenic composition for inducing an antibody that comprises an effector function against an FAM3D-expressing cell, wherein the composition comprises as an active ingredient FAM3D, an immunologically active fragment thereof, or a DNA that can express them; and,
[7] a methodfor inducing anantibodythat comprises aneffector function against an FAM3D-expressing cell, wherein the method comprises administering FAM3D, an immunologically active fragment thereof, or a cell or a DNA that can express them.

The present invention relates to pharmaceutical compositions for damaging FAM3D-expressing cells using antibody effector function, wherein the compositions comprise as an active ingredient an anti-FAM3D antibody. The present invention also relates to uses of an anti-FAM3D antibody to produce pharmaceutical compositions for damaging FAM3D-expressing cells using the anti-FAM3D antibody effector function. The pharmaceutical compositions of the present invention comprise anti-FAM3D antibodies and pharmaceutically acceptable carriers. The present inventors used cDNA microarrays for gene expression analysis of lung cancer cells and normal cells collected from lung cancer patients.

A number of genes with specifically enhanced expression in lung cancer cells were subsequently identified. Of these genes with altered expression in lung cancer cells, genes with low levels of expression in major organs were selected as candidate target genes for lung cancer therapies. By selecting genes with low levels of expression in major organs, it was thought that the danger of side effects could be avoided. Among the proteins encoded by the genes selected in this way, anti-FAM3D antibodies were confirmed to have effector functions against FAM3D-expressing cells, thus completing the invention.

The findings obtained by the present inventors show that, in a forced expression system, FAM3D tagged with c-myc-His was localized in cytoplasmic granules, Golgi bodies, and cytoplasmic membrane. In addition, FAM3D secretion into the culture medium was confirmed using Western blotting, and FAM3D was thus thought to be a secretory protein.

The FAM3D gene encodes an amino acid sequence expected to comprise a signal peptide at its N-terminal. As mentioned above, this protein was observed to be chiefly localized in cytoplasmic granules, Golgi bodies, and the cytoplasmic membrane, and thus it was thought to be a secretory protein. In addition, the low expression level of this gene in major organs, and its high expression in non-small cell cancer cells, suggested that FAM3D is useful as a clinical marker and therapeutic target. However, anti-FAM3D antibodies that display effector function in FAM3D-expressing cells are unknown.

Conditions required for destroying cancer cells using effector function are, for example, the following:
Expression of large numbers of antigenic molecules on the membrane surface of cancer cells,
Uniform distribution of antigens within cancerous tissues,
Lingering of antigens bound to antibodies on the cell surface for a long time.

More specifically, for example, antigens recognized by antibodies must be expressed on the surface of the cell membrane. In addition, it is preferable that the ratio of antigen-positive cells is as high as possible in cells forming cancerous tissues. In an ideal situation, all cancer cells are antigen-positive. When antigen-positive and negative cells are mixed in cancer cell populations, the clinical therapeutic effect of the antibodies may not be expected.

Usually, when as many molecules as possible are expressed on the cell surface, potent effector functions can be expected. It is also important that antibodies bound to antigens are not taken up into cells. Some receptors are taken up into cells (endocytosis) after binding to a ligand. Equally, antibodies bound to cell surface antigens can also be taken up into the cell. This kind of phenomenon, whereby antibodies are taken up into cells, is called internalization. When internalization occurs, the antibody constant (Fc) region is taken up into the cell. However, cells or molecules essential to effector function are outside the antigen-expressing cells. Thus, internalization inhibits antibody effector function. Therefore, when expecting antibody effector function, it is important to select an antigen that causes less antibody internalization. The present inventors revealed for the first time that FAM3D is a target antigen possessing such a property.

“Effector function” in the present invention refers to cytotoxicity involved with the Fc regions of antibodies. Alternatively, functions that drive the effect whereby the Fc regions of antibodies bound to antigens damage cells comprising those antigens, can also be referred to as antibody effector function. Specifically, Antibody Dependent Cell-mediated Cytotoxicity (ADCC), Complement Dependent Cytotoxicity (CDC), and neutralizing activity are known as antibody effector functions. Each function is described below.

Antibody Dependent Cell-mediated Cytotoxicity (ADCC):

Cells exist which comprise Fc receptors specific to the Fc region of immunoglobulin classes IgG, IgE, or IgA. Cells that comprise a corresponding Fc receptor recognize and bind to antibodies bound to cell membranes or so on. For example, an IgG class antibody is recognized by Fc receptors on T cells, NK cells, neutrophils, and macrophages. These cells bind to and are activated by the Fc region of IgG class antibodies, and express cytotoxicity against cells to which these antibodies have bound. Cells which acquire cytotoxicity via antibody effector function are called effector cells. ADCC may be divided based on the type of effector cell, as follows:
ADMC: IgG-dependent macrophage-mediated cytotoxicity, and
ADCC: IgG-dependent NK-cell-mediated cytotoxicity.

There is no limitation on types of effector cells in the ADCC of the present invention. In other words, the ADCC of the present invention also comprises ADMC, where macrophages are the effector cells.

Antibody ADCC has been indicated to compose an important mechanism of the anti-tumor effects, particularly in cancer therapies that use antibodies (Nature Med., 6: 443-446, 2000). For example, a close relationship between the therapeutic effect of anti-CD20 antibody chimeric antibodies and ADCC has been reported (Blood, 99: 754-758, 2002). Thus ADCC is also particularly important among antibody effector functions in the present invention.

For example, ADCC is thought to be an important mechanism in the anti-tumor effects of Rituxan, Herceptin, and so on, for which clinical application has already begun. Rituxan and Herceptin are therapeutic agents for non-Hodgkin's lymphoma and metastatic breast cancer, respectively.

At present, the mechanism for ADCC-mediated cytotoxicity is roughly explained as follows: effector cells, which are bridged to target cells via antibodies bound to the cell surface, are thought to induce target cell apoptosis by transmitting some sort of lethal signal to the target cells. In any case, antibodies that induce cytotoxicity by effector cells are comprised in the antibodies that comprise effector function of the present invention.

Complement Dependent Cytotoxicity (CDC):

The Fc regions of immunoglobulins bound to antigens are known to activate complementary pathways. It has also been revealed that the activation pathway may differ depending on the class of immunoglobulin. For example, of the human antibodies, IgM and IgG activate the classical pathway. On the other hand, IgA, IgD, and IgE do not activate this pathway. The activated complements produce, via a number of reations, a C5b-9 membrane attack complex (MAC) comprising cell membrane-damaging activity. MACs generated in this way are thought to damage viral particles and cell membranes, independently of effector cells. The mechanism for MAC-mediated cytotoxicity is based on the following. MACs comprise a strong binding affinity for cell membranes. MACs bound to a cell membrane open a hole in the cell membrane, making it easy for water to flow in and out of the cell. As a result, the cell membrane is destabilized, or the osmotic pressure is changed, and the cell is destroyed. Cytotoxicity due to an activated complement only extends to membrane close to the antibody which has bound the antigen. For this reason, MAC-mediated cytotoxicity is dependent on antibody specificity. ADCC and CDC can express cytotoxicity independent of each other. However, in practice, these cytotoxicities may function in composite in living bodies.

Neutralizing Activity:

Antibodies exist which have the function of depriving infectivity of pathogens and activity of toxins. Antibody-mediated neutralization can be achieved by binding of an antigenic variable region to an antigen, or can require complement mediation. For example, in some cases, anti-viral antibodies demand complement mediation in order to deprive a virus of its infectivity. Fc regions are essential to the participation of complements. Thus, such antibodies comprise effector function that requires Fc for neutralizing viruses and cells.

In the present invention, effector function can also be explained as a role that determines the biological activity triggered by antigen recognition of an antibody. Herein, preferable target cells are cancer cells. In addition, effector cell functions carried out by the Fc regions of various antibodies rely heavily on antibody class. The Fc region of IgG, IgE, and IgA class antibodies each binds to a specific Fc receptor, and, for example, activates cells that have Fc receptors, and functions in intercellular antibody transport. In particular, IgG class antibodies activate effector cells via Fc receptors on these cells, and then kill target cells to which the variable regions of the antibodies are bound. This is called antibody-dependent cell-mediated cytotoxicity (ADCC). In ADCC, T cells, NK cells, neutrophils, macrophages, or such function as effector cells. On the other hand, the function of activating complement is limited to IgM and IgG class antibodies. Particularly, the function of lysing cells to which antibody variable regions are bound is called complement-dependent cytotoxicity (CDC).

Of these, preferable effector functions herein are either ADCC or CDC, or both. The present invention is based on the finding that anti-FAM3D antibodies bind to FAM3D-expressing cells, and then express effector function.

The present invention also relates to methods for damaging FAM3D-expressing cells, which comprise the following steps:
1) contacting the FAM3D-expressing cells with anti-FAM3D antibodies, and
2) using the effector function of the antibodies which have bound to the FAM3D-expressing cells to damage the cells.

In the methods for damaging cells or pharmaceutical compositions of the present invention, any cell can be used as an FAM3D-expressing cell. For example, lung cancer cells are preferable as the FAM3D-expressing cells of the present invention. Of these, non-small cell lung cancer (NSCLC) cells are preferable. Cells and antibodies can be contacted in vivo or in vitro. When targeting in vivo lung cancer cells as the FAM3D-expressing cells, the methods of the present invention are in fact therapeutic methods or preventative methods for lung cancers. Specifically, the present invention provides therapeutic methods for lung cancers which comprise the following steps:
1) administering an antibody that binds FAM3D to a lung cancer patient, and
2) damaging lung cancer cells using the effector function of the antibody bound to those cells.

FAM3D, which the present inventors identified as a gene overexpressed in lung cancers, was confirmed to be specifically expressed on the surface of lung cancer cells, and to be hardly expressed in the cells of organs essential for maintaining life. In addition, anti-FAM3D antibodies can be specific to antigens on the surface of lung cancer cells, and their effector function may induce immune system cells to exert cytotoxicity against cancer cells.

The present inventors confirmed that antibodies binding FAM3D effectively damage FAM3D-expressing cells, in particular, lung cancer cells using effector function. The present inventors also confirmed that FAM3D is highly expressed in lung cancer cells, with a high probability. In addition, FAM3D expression levels in normal tissues are low. Putting this information together, methods of lung cancer therapy where FAM3D is administered can be effective, with little danger of side effects.

The antibodies of the present invention are not limited so long as they comprise a desired effector function. For example, antibodies comprising the Fc region of IgA, IgE, or IgG are essential for expressing ADCC. Equally, the antibody Fc region of IgM or IgG is preferable for expressing CDC. Therefore, human-derived antibodies belonging to these classes are preferable in the present invention. Human antibodies can be acquired using antibody-producing cells harvested from humans, or chimeric animals transplanted with human antibody genes (Cloning and Stem Cells., 4: 85-95, 2002).

Furthermore, antibody Fc regions can link with arbitrary variable regions. Specifically, chimeric antibodies wherein the variable regions of different animal species are bound to human constant regions are known. Alternatively, a human-human chimeric antibody can also be acquired by binding human-derived variable regions to arbitrary constant regions. In addition, CDR graft technology, where complementarity determining regions (CDRs) composing human antibody variable regions are replaced with CDRs of heterologous antibodies, is also known (“Immunoglobulin genes”, Academic Press (London), pp 260-274, 1989; Proc. Natl. Acad. Sci. USA. 91: 969-973, 1994). By replacing CDRS, antibody binding specificity is replaced. That is, human FAM3D will be recognized by humanized antibodies in which the CDR of human FAM3D-binding antibodies has been transferred. The transferred antibodies can also be called humanized antibodies. Antibodies thus-obtained and equipped with an Fc region essential to effector function can be used as the antibodies of the present invention, regardless of the origin of their variable regions. For example, antibodies comprising a human IgG Fc are preferable in the present invention, even if their variable regions comprise an amino acid sequence derived from an immunoglobulin of another class or another species.

The antibodies of the present invention may be monoclonal antibodies or polyclonal antibodies. Even when administering to humans, human polyclonal antibodies can be derived using the above-mentioned animals transferred with a human antibody gene. Alternatively, immunoglobulins which have been constructed using genetic engineering techniques, such as humanized antibodies, human-non-human chimeric antibodies, and human-human chimeric antibodies, can be used. Furthermore, methods for obtaining human monoclonal antibodies by cloning human antibody-producing cells are also known.

FAM3D, or a fragment comprising its partial peptide, can be used as immunogens to obtain the antibodies of the present invention. The FAM3D of the present invention can be derived from an arbitrary species, preferably from a mammal such as a human, mouse, or rat, and more preferably from a human. The human FAM3D nucleotide sequence and amino acid sequence are known (NM_{—}138805). The cDNA nucleotide sequence of FAM3D is described in SEQ ID NO: 1, and the amino acid sequences coded by that nucleotide sequence is described in SEQ ID NO: 2. One skilled in the art can routinely isolate genes comprising the provided nucleotide sequence, preparing a fragment of the sequence as required, and obtain a protein comprising the target amino acid sequence.

For example, the gene coding the FAM3D protein or its fragment can be inserted into a known expression vector, and used to transform host cells. The desired protein, or its fragment, can be collected from inside or outside host cells using arbitrary and standard methods, and can also be used as an antigen. In addition, proteins, their lysates, and chemically-synthesized proteins can be used as antigens. Furthermore, cells expressing the FAM3D protein or a fragment thereof can themselves be used as immunogens.

When using a peptide fragment as the FAM3D immunogen, it is particularly preferable to select an amino acid sequence which comprises a region predicted to be an extra-cellular domain. The existence of a signal peptide is predicted from positions 1 to 16 on the N-terminal of FAM3D. Thus, for example, a region other than the N-terminal signal peptide (16 amino acid residues) is preferred as the immunogen for obtaining the antibodies of the present invention. That is to say, antibodies that bind to FAM3D extra-cellular domains are preferred as the antibodies of the present invention.

Therefore, preferable antibodies in the present invention are antibodies equipped with an Fc essential to effector function, and a variable region that can bind to an extracellular FAM3D domain. When aiming for administration to humans, it is preferable to be equipped with an IgG Fc.

An arbitrary mammal can be immunized with such an antigen. However, it is preferable to consider compatibility with parent cells used in cell fusion. Generally, rodents, lagomorphs, or primates are used.

Rodents include, for example, mice, rats, and hamsters. Lagomorphs include, for example, rabbits. Primates include, for example, catarrhine (old world) monkeys such as Macaca fascicularis, Macaca mulatta, Sacred baboons, and chimpanzees.

Methods for immunizing animals with antigens are well known in the field. Intraperitoneal or subcutaneous antigen injections are standard methods for immunizing mammals. Specifically, antigens can be diluted and suspended in an appropriate amount of phosphate buffered saline (PBS), physiological saline, or so on. As desired, antigen suspensions can be mixed with an appropriate amount of a standard adjuvant such as Freund's complete adjuvant, and administered to mammals after emulsification. Subsequently, it is preferable that antigens mixed with an appropriate amount of Freund's incomplete adjuvant are administered in multiple doses every four to 21 days. An appropriate carrier can also be used for immunization. After carrying out immunization as outlined above, standard methods can be used to examine serum for an increase in the desired antibody level.

Polyclonal antibodies against the FAM3D protein can be prepared from immunized mammals whose serum has been investigated for an increase in the desired antibodies. This can be achieved by collecting blood from these animals, or by using an arbitrary, usual method to isolate serum from their blood. Polyclonal antibodies comprise serum that comprises polyclonal antibodies, and fractions that comprise polyclonal antibodies which can be isolated from serum. IgG and IgM can be prepared from fractions that recognize FAM3D protein by using, for example, an affinity column coupled to FAM3D protein, and then further purifying this fraction using protein A or protein G columns. In the present invention, antiserum can be used as is as polyclonal antibodies. Alternatively, purified IgG, IgM, or such can also be used.

To prepare monoclonal antibodies, immunocytes are collected from mammals immunized with antigens, investigated for the increase of the desired antibody level in serum (as above), and applied in cell fusion. Immunocytes for use in cell fusion preferably come from the spleen. Other preferred parent cells for fusion with the above immunogens include, for example, mammalian myeloma cells, and more preferably, myeloma cells that have acquired properties for selection of fusion cells by pharmaceutical agents.

The above immunocytes and myeloma cells can be fused using known methods, for example the methods of Milstein et al. (Galfre, G. and Milstein, C., Methods. Enzymol, 1981, 73, 3-46).

Hybridomas produced by cell fusion can be selected by culturing in a standard selective medium such as HAT medium (medium comprising hypoxanthine, aminopterin, and thymidine). Cell culture in HAT medium is usually continued for several days to several weeks, a period sufficient enough to kill all cells other than the desired hybridomas (unfused cells). Standard limiting dilutions are then carried out, and hybridoma cells that produce the desired antibodies are screened and cloned.

Non-human animals can be immunized with antigens for preparing hybridomas in the above method. In addition, human lymphocytes from cells infected with EB virus or such, can be immunized in vitro using proteins, cells expressing proteins, or suspensions of the same. The immunized lymphocytes are then fused with human-derived myeloma cells able to divide unlimitedly (U266 and so on), thus obtaining hybridomas that produce the desired human antibodies which can bind the protein (Unexamined Published Japanese Patent Application No. (JP-A) Sho 63-17688).

The obtained hybridomas are then transplanted to mice abdominal cavities, and ascites are extracted. The obtained monoclonal antibodies can be purified using, for example, ammonium sulfate precipitation, protein A or protein G columns, DEAE ion exchange chromatography, or affinity columns coupled to the proteins of the present invention. The antibodies of the present invention can be used not only in purifying and detecting the proteins of the present invention, but also as candidates for agonists and antagonists of the proteins of the present invention. These antibodies can also be applied to antibody therapies for diseases related to the proteins of the present invention. When the obtained antibodies are administered to human bodies (antibody therapy), human antibodies or humanized antibodies are preferred due to their low immunogenicity.

For example, transgenic animals comprising a repertoire of human antibody genes can be immunized with antigens selected from proteins, protein-expressing cells, or suspensions of the same. Antibody-producing cells are then recovered from the animals, fused with myeloma cells to yield hybridomas, and anti-protein human antibodies can be prepared from these hybridomas (see International Publication No. 92-03918, 93-2227, 94-02602, 94-25585, 96-33735, and 96-34096).

Alternatively, immunocytes such as immunized lymphocytes that produce antibodies, can be immortalized using cancer genes, and used to prepare monoclonal antibodies.

Monoclonal antibodies obtained in this way can be prepared using methods of genetic engineering (for example, see Borrebaeck, C. A. K. and Larrick, J. W., Therapeutic Monoclonal Antibodies, MacMillan Publishers, UK, 1990). For example, recombinant antibodies can be prepared by cloning DNAs that encode antibodies from immunocytes such as hybridomas or immunized lymphocytes that produce antibodies; then inserting these DNAs into appropriate vectors; and transforming these into host cells. Recombinant antibodies prepared as above can also be used in the present invention.

The antibodies can be modified by binding with a variety of molecules such as polyethylene glycols (PEGs). Antibodies modified in this way can also be used in the present invention. Modified antibodies can be obtained by chemically modifying antibodies. These kinds of modification methods are conventional to those skilled in the art. The antibodies can also be modified by other proteins. Antibodies modified by protein molecules can be produced using genetic engineering. That is, target proteins can be expressed by fusing antibody genes with genes that code for modification proteins. For example, antibody effector function may be enhanced on binding with cytokines or chemokines. In fact, the enhancement of antibody effector function for proteins fused with IL-2, GM-CSF, and such has been confirmed (Human Antibody, 10: 43-49, 2000). IL-2, IL-12, GM-CSF, TNF, eosinophil chemotactic substance (RANTES) and so on can be included in cytokines or chemokines that enhance effector function.

Alternatively, antibodies of the present invention can be obtained as chimeric antibodies which comprise a non-human antibody-derived variable region and a human antibody-derived constant region, or as humanized antibodies which comprise a non-human antibody-derived complementarity determining region (CDR), a human antibody-derived framework region (FR), and a constant region. Such antibodies can be produced using known methods.

Antibodies obtained as above can be purified until uniform. For example, antibodies can be purified or separated according to general methods used for purifying and separating proteins. For example, antibodies can be separated and isolated using appropriately selected combinations of column chromatography, comprising but not limited to affinity chromatography, filtration, ultrafiltration, salt precipitation, dialysis, SDS polyacrylamide gel electrophoresis, isoelectric focusing, and so on (Antibodies: A Laboratory Manual, Harlow and David, Lane (edit.), Cold Spring Harbor Laboratory, 1988).

Protein A columns and Protein G columns can be used as affinity columns. Exemplary protein A columns in use include Hyper D, POROS, and Sepharose F. F (Pharmacia).

Exemplary chromatography (excluding affinity chromatography) include ion exchange chromatography, hydrophobic chromatography, gel filtration, reverse phase chromatography, and adsorption chromatography (“Strategies for Protein Purification and Characterization: A Laboratory Course Manual” Daniel R. Marshak et al., Cold Spring Harbor Laboratory Press, 1996). The chromatography can be performed according to the procedure of liquid phase chromatographies such as HPLC or FPLC.

For example, the antigen-binding activity of the antibodies of the present invention can be measured by using absorbance measurements, enzyme linked immunosorbent assays (ELISA), enzyme immunoassays (EIA), radioimmunoassays (RIA) and/or immunofluorescence methods. In ELISA, an antibody of the present invention is immobilized on a plate, a protein of the present invention is added to the plate, and then a sample comprising the desired antibody such as the culture supernatant of cells that produce the antibody or purified antibody is added. A secondary antibody that recognizes the primary antibody and has been tagged with an enzyme such as alkaline phosphatase is then added, and the plate is incubated. After washing, an enzyme substrate such as p-nitrophenyl phosphate is added to the plate, absorbance is measured, and the antigen-binding activity of the samples is evaluated. Protein fragments (C-terminal or N-terminal fragments, and such) can be used in the same way as proteins. The binding activity of the antibodies of the present invention can be evaluated using BIAcore (Pharmacia).

In addition, by following the methods outlined in the Examples, antibody effector function can also be evaluated. For example, target FAM3D-expressing cells are incubated with effector cells in the presence of an antibody whose effector function is to be evaluated. If target cell destruction is detected, the antibody can be confirmed to comprise effector function that induces ADCC. The level of observed target cell destruction, in the absence of either antibodies or effector cells, can be compared as a control with the level of effector function. Cells which clearly express FAM3D can be used as the target cells. Specifically, a variety of cell lines confirmed to express FAM3D in the Examples can be used. These cell lines can be obtained from cell banks. In addition, monoclonal antibodies which comprise more powerful effector function can be selected.

In the present invention, anti-FAM3D antibodies can be administered to humans or other animals as pharmaceutical agents. In the present invention, animals other than humans to which the antibodies can be administered include mice, rats, guinea pigs, rabbits, chickens, cats, dogs, sheep, pigs, cows, monkeys, baboons, and chimpanzees. The antibodies can be directly administered to subjects, and in addition, can be formulated into dosage forms using known pharmaceutical formulation methods. For example, depending on requirements, they can be parenterally administered in an injectable form such as a sterile solution or suspension with water or other arbitrary pharmaceutically acceptable fluid. For example, this kind of compounds can be mixed with acceptable carriers or solvents, specifically sterile water, physiological saline, vegetable oils, emulsifiers, suspension agents, surfactants, stabilizers, flavoring agents, excipients, solvents, preservatives, binding agents and the like, into a generally accepted unit dosage essential for use as a pharmaceutical agent.

Other isotonic solutions comprising physiological saline, glucose, and adjuvants (such as D-sorbitol, D-mannose, D-mannitol, and sodium chloride) can be used as the injectable aqueous solution. They can also be used with appropriate solubilizers such as alcohols, specifically ethanols and polyalcohols (for example, propylene glycols and polyethylene glycol), and non-ionic surfactants (for example polysorbate 80™ or HCO-50).

Sesame oils or soybean oils can be used as an oleaginous solution, and benzyl benzoate or benzyl alcohols can be used with them as a solubilizer. Buffer solutions (phosphate buffers, sodium acetate buffers, or so on), analgesics (procaine hydrochloride or such), stabilizers (benzyl alcohol, phenols, or so on), and antioxidants can be used in the formulation. The prepared injections can be packaged into appropriate ampules.

In the present invention, the anti-FAM3D antibodies can be administered to patients, for example, intraarterially, intravenously, or percutaneously, or intranasally, transbronchially, locally, or intramuscularly. Intravascular (intravenous) administration by drip or injection is an example of a general method for systematic administration of antibodies to lung cancer patients. Methods of locally concentrating antibody agents to the primary focus or metastatic focus in the lungs include local injection using a bronchoscope (bronchoscopy) and local injection under CT guidance or with thoracoscopy. In addition, methods in which an intraarterial catheter is inserted near a vein that supplies nutrients to cancer cells to locally inject anti-cancer agents such as antibody agents, are effective as local control therapies for metastatic focuses as well as primary focuses of lung cancer.

Although dosage and administration methods vary according to patient body weight and age, and administration method, these can be routinely selected by one skilled in the art. In addition, DNA encoding an antibody can be inserted into a vector for gene therapy, and the vector can be administered for therapy. Dosage and administration methods vary according to patient body weight, age, and condition, however, one skilled in the art can select these appropriately.

Anti-FAM3D antibodies can be administered to living bodies in an amount such that cytotoxicity based on effector function against FAM3D-expressing cells can be confirmed. For example, although there is a certain amount of difference depending on symptoms, anti-FAM3D antibody dosage is 0.1 mg to 250 mg/kg per day. Usually, the dosage for an adult (of weight 60 kg) is 5 mg to 17.5 g/day, preferably 5 mg to 10 g/day, and more preferably 100 mg to 3 g/day. The dosage schedule is from one to ten times over a two to ten day interval, and for example, progress is observed after a three to six times administration.

In addition, the present invention provides immunogenic compositions for inducing antibodies comprising effector functions against FAM3D-expressing cells, where the compositions comprise as an active ingredient FAM3D or an immunologically active FAM3D fragment, or a DNA or cell which can express the same. Alternatively, the present invention relates to uses of FAM3D or an immunologically active FAM3D fragment, or a DNA or cell which can express the same in the production of immunogenic compositions for inducing antibodies comprising effector functions against FAM3D-expressing cells.

The administration of anti-FAM3D antibodies damages cancer cells by the effector function of those antibodies. Thus, if FAM3D antibodies can be induced in vivo, therapeutic effects equivalent to the antibody administration can be achieved. When administering immunogenic compositions comprising antigens, target antibodies can be induced in vivo. The immunogenic compositions of the present invention make vaccine therapy against FAM3D-expressing cells possible. Thus, the immunogenic compositions of the present invention are effective as, for example, vaccine compositions for lung cancer therapies.

The immunogenic compositions of the present invention can comprise FAM3D or an immunologically active FAM3D fragment, as an active ingredient. An immunologically active FAM3D fragment refers to a fragment that can induce anti-FAM3D antibodies which recognize FAM3D and comprise effector function. Below, FAM3D and the immunologically active FAM3D fragment are described as immunogenic proteins. Whether a given fragment induces target antibodies can be determined by actually immunizing an animal, and confirming the activity of the induced antibodies. Antibody induction and the confirmation of its activity can be carried out, for example, using methods described in Examples. For example, fragments comprising an amino acid sequence corresponding to FAM3D position 28 to 172, or FAM3D position 69 to 208, can be used as the immunogens of the present invention.

The immunogenic compositions of the present invention comprise pharmaceutically acceptable carriers as well as immunogenic proteins, the active ingredients. If necessary, the compositions can also be combined with an adjuvant. Killed tuberculosis bacteria, diphtheria toxoid, saponin and so on can be used as the adjuvant.

Alternatively, DNAs coding for the immunogenic proteins, or cells retaining those DNAs in an expressible state, can be used as the immunogenic compositions. Methods for using DNAs expressing the target antigen as immunogens, so-called DNA vaccines, are well known. DNA vaccines can be obtained by inserting a DNA encoding FAM3D or its fragment into an appropriate expression vector.

Retrovirus vectors, adenovirus vectors, adeno-associated virus vectors, Sendai virus vectors or such can be used as the vector. In addition, DNAs in which a DNA encoding an immunogenic protein is functionally connected downstream of a promoter can be directly introduced into cells as naked DNA, and then expressed. Naked DNA can be encapsulated in ribosomes or viral envelope vectors and introduced into cells.

Furthermore, immunogenic protein-expressing cells in which vectors or DNAs that can express the immunogenic proteins can be used as the immunogenic compositions of the present invention. For example, patient blood cells are collected, transformed using a vector that can express the immunogenic proteins, and returned to the patient. Transformed bloods cells produce the immunogenic proteins inside the body of the patient, and induce the target antibodies.

When DNAs encoding the immunogenic proteins, or cells transformed with the same are used as immunogenic compositions of the present invention, they can be combined with immunogenic proteins as well as carrier proteins that enhance their immunogenic properties.

Alternatively, the present invention provides methods for inducing antibodies which comprise effector function against FAM3D-expressing cells, where the methods comprise the step of administering FAM3D, an immunologically active FAM3D fragment, or DNA or cells that can express the same. The methods of the present invention induce antibodies that comprise effector function that damages FAM3D-expressing cells such as lung cancers. As a result, therapeutic effects for lung cancers and so on can be obtained.

Each day, 0.1 mg to 250 mg per kilogram of the immunogenic compositions of the present invention can be administered orally or parenterally. Parenteral administration includes subcutaneous injection and intravenous injection. The administrative dose for a single adult is usually 5 mg to 17.5 g/day, preferably 5 mg to 10 g/day, and more preferably 100 mg to 3 g/day.

All prior references cited herein are incorporated by reference in their entirety.

### BRIEF DESCRIPTION OF THE DRAWINGS

FIG. 1 shows the results of an ADCC assay using anti-FAM3D antibodies. The vertical axis shows cytotoxicity (%), and the horizontal axis shows the ratio of effector cell:target cell (E:T ratio). Cytotoxicity was determined by:

$$\% specific lysis = 100 \times \frac{( experimental cpm - spontaneous cpm )}{( maximum cpm - spontaneous cpm )}$$

Experimental cpm: Supernatant counts measured under each set of conditions
Spontaneous cpm: Background counts
Maximum cpm: Tagged target cell counts

### BEST MODE FOR CARRYING OUT THE INVENTION

Below, the present invention is further explained based on Examples.

Cell Line:

Human lung cancer cell lines were propagated as a monolayer in an appropriate medium with 10% fetal bovine serum. The cell lines used in the experiment are shown in Table 1.

TABLE 1

| | | |
|---|---|---|
| Cell line | Medium | Place obtained |

Lung adenocarcinomas (ADC)

| | | |
|---|---|---|
| PC-14 | RPMI-1640 (10% FBS) | Tokyo Medical |
| | | College |
| LC319 | RPMI-1640 (10% FBS) | Aichi Cancer Center |
| NCI-H1373 | RPMI-1640 (10% FBS) | ATCC (CRL-5866) |

Lung squamous carcinomas (SCC)

| | | |
|---|---|---|
| RERF-LC-AI | DMEM (10% FBS) | RERF |
| EBC-1 | DMEM (10% FBS) | Okayama University |
| NCI-H2170 | RPMI-1640 (10% FBS) | ATCC (CRL-5928) |
| NCI-H226 | RPMI-1640 (10% FBS) | ATCC (CRL-5826) |

Small-cell lung cancers (SCLC)

| | | |
|---|---|---|
| DMS114 | RPMI-1640 (10% FBS) | ATCC (CRL-2066) |
| SBC-3 | DMEM (10% FBS) | Okayama University |
| SBC-5 | EMEM (10% FBS) | Okayama University |

Furthermore, the following cell lines were used in ADCC assays using anti-FAM3D antibodies:
Lung adenocarcinomas (ADC): LC319, PC-14, NCI-H1373
Lung squamous carcinomas (SCC): RERF-LC-AI, EBC-1, NCI-H2170, NCL-H226
Small-cell lung cancers (SCLC): DMS114, SBC-3, SBC-5.
   Antibody Production:

According to standard protocols, individual protein specific antibodies were produced using His-tagged fusion proteins expressed in bacteria as immunogens. These fusion proteins comprised a protein portion that corresponded to one part of the protein (residues 28 to 172, and residues 69 to 208).

Flow Cytometry Analysis:

Cancer cells (1×10⁶) were incubated at 4° C. for one hour with the purified polyclonal antibodies (pAb) or rabbit IgG (the control) Cells were washed with phosphate buffer solution (PBS) and then incubated at 4° C. for 30 minutes in FITC-labeled Alexa Flour 488. The cells were again washed in PBS, analyzed on a flow cytometer (FACScan, Becton Dickinson), and then analyzed by ModFit software (Verity Software House, Inc). Mean fluorescence intensity (MFI) was defined as a ratio of the flow cytometric intensity (intensity by each protein specific antibody/intensity by rabbit IgG).

Using anti-FAM3D-antibodies BB016, FAM3D expression was investigated for LC319, PC-14, NCI-H1373, RERF-LC-AI, EBC-1, NCI-H2170, NCL-H226, DMS114, SBC-3, and SBC-5 cells. As a result, a higher proportion of anti-FAM3D antibodies (BB016) bound to all cells (Table 2) than did rabbit IgG (the control).

TABLE 2

| | | FAM3D expression |
|---|---|---|
| Cell line | Origin | (MFI^{a}) |
| LC319 | ADC^{b} | 14.5 |
| PC-14 | ADC | 5.7 |
| NCI-H1373 | ADC | 5.0 |
| RERF-LC-AI | SCC^{b} | 5.6 |
| EBC-1 | SCC | 2.9 |
| HCI-H2170 | SCC | 19.6 |
| HCI-H226 | SCC | 4 |
| DMS114 | SCLC^{d} | 5.9 |
| SBC-3 | SCLC | 6.3 |
| SBC-5 | SCLC | 29.9 |

^{a}Mean fluorescence intensity
^{b}Lung adenocarcinoma
c Lung squamous carcinoma
^{d}Small-cell lung cancer

ADCC Assays:

Target cells were labeled with ⁵¹Cr 100 μCi at 37° C. for one hour, and these cells were mixed every ten minutes and maintained as a suspension. Target cancer cells were washed two times before being added to the assay, and cells were then plated on 96-well U-bottom plates (2×10⁴ cells/well). Human peripheral blood mononuclear cells (PMBC) were harvested from a healthy person, separated using Ficoll-Paque (Amersham Biosciences) density gradient centrifugation, and then used as effector cells. Target cancer cells (T) and effector cells (E) were incubated in 96-well U-bottom plates at various E:T ratios (50:1, 25:1, 12.5:1, and 6.25:1) with BB016 anti-FAM3D antibody (2 μg/well) or control antibody Herceptin (2 μg/well, Roche). This incubation was carried out in triplicate, in 200 μL of AIM-V medium (Life Technologies, Inc), at 37° C. for six hours. The ADCC effects of anti-FAM3D antibody (BB016) for SBC-5 cells were evaluated based on the value when the radioactivity of supernatant (70 μL) was measured with a gamma counter. The proportion of specific lysis was calculated according to the following formula:

$$\% specific lysis = 100 \times \frac{( experimental cpm - spontaneous cpm )}{( maximum cpm - spontaneous cpm )}$$

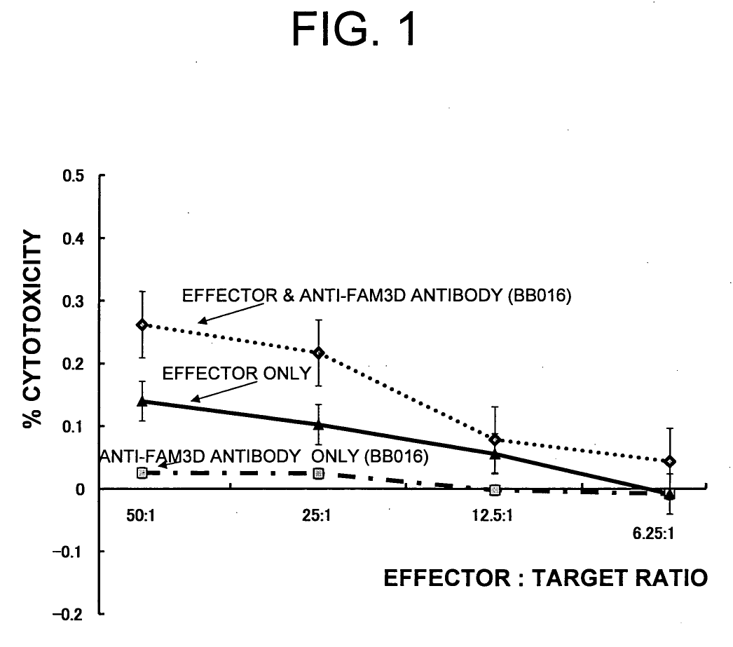

Control assays were carried out by incubating target cells with only BB016anti-FAM3D antibody or only effector cells. Herceptin was used as a control in several experiments. Direct cell damage of SBC-5 cells by BB016 anti-FAM3D antibody itself was not observed. However, BB016 induced ADCC in SBC-5 cells that over-expressed FAM3D (FIG. 1).

### INDUSTRIAL APPLICABILITY

The present invention revealed that FAM3D-expressing cells can be damaged by antibody cytotoxicity. FAM3D was identified by the present inventors as a gene strongly expressed in lung cancers. Thus, lung cancer therapies are possible using antibodies that bind to FAM3D. Results actually confirmed by the present inventors show cytotoxicity due to the effect of ADCC in lung cancer cell lines, in the presence of FAM3D antibodies.

1. A pharmaceutical composition comprising an anti-FAM3D antibody as an active ingredient, wherein the compound is for damaging an FAM3D-expressing cell using antibody effector function.

2. The pharmaceutical composition of claim 1, wherein the FAM3D-expressing cell is a lung cancer cell.

3. The pharmaceutical composition of claim 1, wherein the anti-FAM3D antibody is a monoclonal antibody.

4. The pharmaceutical composition of claim 1, wherein the antibody effector function is either antibody-dependent cytotoxicity or complement-dependent cytotoxicity, or both.

5. A method for damaging an FAM3D-expressing cell, comprising the steps of:
a) contacting the FAM3D-expressing cell with an anti-FAM3D antibody, and
b) damaging the FAM3D-expressing cell with the effector function of the antibody that has bound to the cell.

6. An immunogenic composition for inducing an antibody that comprises an effector function against an FAM3D-expressing cell, wherein the composition comprises, as an active ingredient, FAM3D, an immunologically active fragment thereof, or a DNA that can express them.

7. A method for inducing an antibody that comprises an effector function against an FAM3D-expressing cell, wherein the method comprises administering FAM3D, an immunologically active fragment thereof, or a cell or a DNA that can express them.


SEQUENCE LISTING (2 sequences)

SEQ ID NO: 1 - DNA, 1322 bp, Homo sapiens
  CDS (298)..(972): FAM3D
tcctcaaaggaggggcagagcctgcgcagggcaggagcagctggcccactggcggcccgcaacactccgtctcaccctctgggcccactgcatctagaggagggccgtctgtgaggccactacccctccagcaactgggaggtgggactgtcagaagctggcccagggtggtggtcagctgggtcagggacctacggcacctgctggaccacctcgccttctccatcgaagcagggaagtgggagcctcgagccctcgggtggaagctgaccccaagccacccttcacctggacaggatgagagtgtcaggtgtgcttcgcctcctggccctcatctttgccatagtcacgacatggatgtttattcgaagctacatgagcttcagcatgaaaaccatccgtctgccacgctggctggcagcctcgcccaccaaggagatccaggttaaaaagtacaagtgtggcctcatcaagccctgcccagccaactactttgcgtttaaaatctgcagtggggccgccaacgtcgtgggccctactatgtgctttgaagaccgcatgatcatgagtcctgtgaaaaacaatgtgggcagaggcctaaacatcgccctggtgaatggaaccacgggagctgtgctgggacagaaggcatttgacatgtactctggagatgttatgcacctagtgaaattccttaaagaaattccggggggtgcactggtgctggtggcctcctacgacgatccagggaccaaaatgaacgatgaaagcaggaaactcttctctgacttggggagttcctacgcaaaacaactgggcttccgggacagctgggtcttcataggagccaaagacctcaggggtaaaagcccctttgagcagttcttaaagaacagcccagacacaaacaaatacgagggatggccagagctgctggagatggagggctgcatgcccccgaagccattttagggtggctgtggctcttcctcagccaggggcctgaagaagctcctgcctgacttaggagtcagagcccggcaggggctgaggaggaggagcagggggtgctgcgtggaaggtgctgcaggtccttgcacgctgtgtcgcgcctctcctcctcggaaacagaaccctcccacagcacatcctacccggaagaccagcctcagagggtccttctggaaccagctgtctgtggagagaatggggtgctttcgtcagggactgctgacggctggtcctgaggaaggacaaactgcccagacttgagcccaattaaattttatttttgctggttttgaatgaaaaaaaaaaaaaaa

SEQ ID NO: 2 - protein, 224 aa, Homo sapiens
  FAM3D
MRVSGVLRLLALIFAIVTTWMFIRSYMSFSMKTIRLPRWLAASPTKEIQVKKYKCGLIKPCPANYFAFKICSGAANVVGPTMCFEDRMIMSPVKNNVGRGLNIALVNGTTGAVLGQKAFDMYSGDVMHLVKFLKEIPGGALVLVASYDDPGTKMNDESRKLFSDLGSSYAKQLGFRDSWVFIGAKDLRGKSPFEQFLKNSPDTNKYEGWPELLEMEGCMPPKPF